(19) United States
(12) Patent Application Publication
(10) Pub. No.: US 2025/0127947 A1
(43) Pub. Date: Apr. 24, 2025

(54) DEVICE FOR VERIFICATION OF BIOLOGICAL INDICATORS IN A STERILISATION OR DECONTAMINATION CHAMBER FOR PRODUCTION

(71) Applicant: FEDEGARI AUTOCLAVI S.P.A., Pavia (IT)

(72) Inventors: Giuseppe FEDEGARI, Pavia (IT); Paolo FEDEGARI, Celerina/Schlarigna (CH)

(73) Assignee: FEDEGARI AUTOCLAVI S.P.A., Pavia (IT)

(21) Appl. No.: 18/836,107

(22) PCT Filed: Feb. 6, 2023
(86) PCT No.: PCT/IB2023/051035

§ 371 Date: Aug. 6, 2024

(30) Foreign Application Priority Data
Feb. 7, 2022 (IT) 102022000002126

(51) Int. Cl. A61L 2/28

(52) U.S. Cl. CPC A61L 2/28

(57) ABSTRACT

The present invention relates to an integration kit for a normal sterilisation machine (1) comprising a sterilisation chamber (2) configured to contain the products to be sterilised, the integration kit comprising:-an indicator loading element (10) which is insertable and removable from said sterilisation chamber (2) following a sterilisation process; and-a bioindicator guide channel (20) configured to removably receive said indicator loading element (10) therein, the guide channel (20) having a first portion (22a) connectable outside of the sterilisation chamber (2) through a through-hole and a second portion (22b, 22c) which, during use, extends inside the sterilisation chamber (2) and which can be selectively put in fluid communication therewith so as to allow the passage therefrom and thereto of a sterilising agent present inside the sterilisation chamber (2). The invention also relates to a method for characterising biological indicators used in product sterilisation processes.

The present invention relates to the verification of biological indicators used for controlling sterilisation processes in the pharmaceutical, food, and healthcare industries and the like. As is well known, the purpose of sterilisation processes is to destroy the microbial load contained inside on the surfaces of the products subject to the treatment.

This test is typically performed according to two methods which complement each other and are applicable in parallel.

The first method is of a physical type (measurement of physical parameters such as temperature, pressure, concentration of the sterilising agent) and, in the cases of moist heat processes (such as, for example saturated steam processes), it aims to assure that even the coldest point of the load has reached the minimum sterilisation temperature.

The aforesaid values are the ones the operator deems suitable for the complete destruction of the microbial load potentially present on the product.

The detection of the minimum physical sterilisation parameters by the sensors may, in some cases, not be sufficient to assure the actual sterilisation of the product: in fact, both the sensor position and the type of physical parameter detected always provide indirect or incomplete information about the overall performance of the process.

In the case of steam sterilisation, for example, the reaching of the sterilisation temperature does not necessarily imply the presence of saturated steam in (direct or indirect) contact with the microorganisms to be inactivated, which is an essential condition for the destruction of the microbial load.

The second test system alluded to above is based on the use of so-called biological indicators or bioindicators; these are preparations of special (non-pathogenic) spore-forming microorganisms that are particularly resistant to sterilisation.

Starting from the assumption that bioindicators are prepared so as to be more resistant to the action of the sterilising agent (which can be for example steam or hot air in thermal processes, or ethylene oxide or hydrogen peroxide in “cold” chemical processes) than the microorganisms potentially present on the product to be sterilised, if, at the end of the treatment, one obtains the destruction of the bioindicators, all the more reason why the product as well can be considered sterilised.

Bioindicators are typically produced by specialised firms in various forms, for example in aqueous suspensions contained in phials or vials, or deposited on different substrates (textile, paper, elastomeric or of another nature) and then dried.

Bioindicators are identified by specific parameters: the type of spores used, the number NO of spores contained in the bioindicator, the rate of destruction D of the spores as a function of the sterilisation temperature, and the variation z in the aforesaid rate as a function of the variation in the sterilisation temperature.

The knowledge and thus determination of such parameters is fundamental in order to be able to use the bioindicator.

However, whereas the determination of NO is within the capabilities of any microbiologist, the determination of D and z is anything but easy and requires laborious procedures with scarcely repetitive results.

At the end of the thermal cycle, the samples of bioindicators are analysed in order to count how many spores survived and then the values of D and z are calculated.

The apparatus used for the verification of biological indicators, called BIERs (“Biological Indicator Evaluator Resistometers”), are complex, delicate and very costly, besides being scarcely available in the market.

BIERs are specially designed machines that use very small sterilisation chambers in order to reach the sterilisation conditions under which it is desired to verify the indicators in very short periods of time.

Essentially, BIER apparatus were developed for the sole purpose of enabling a measurement of the reduction in the microbial load under the sterilisation conditions with variations in the duration of exposure and not during the transitory phases (pre- and post-conditioning phases, during which a progressive reduction also occurs).

For the user, moreover, BIER apparatus represent an extra piece of equipment to buy, install, maintain and periodically evaluate, in addition to the ones normally in use. In this context, the technical task at the basis of the present invention is to propose an integration kit for a normal sterilisation machine that overcomes the above-mentioned drawbacks of the prior art.

In particular, it is an object of the present invention to provide a device to be integrated into a sterilisation machine so as to enable biological indicators to be characterised, without altering the machine's functionality.

A further object of the present invention is to enable a normal sterilisation or decontamination machine to be used both as a BIER and as a normal steriliser.

A further object of the present invention is to allow biological indicators to be characterised in a precise and efficient manner.

Yet another object of the present invention is to enable a rapid conversion from a normal autoclave to a BIER and vice versa without the use of special tools (in less than 5 minutes), and without compromising the performance and reliability of the steriliser.

Another object of the present invention is to provide a normal sterilisation machine that is easy to use and has a simple bioindicator loading and unloading mechanism.

Finally, a further object of the present invention is to enable the characterisation of biological indicators using a normal autoclave at considerably lower costs than those associated with the use of a BIER.

The stated technical task and specified objects are substantially achieved by an accessory device for the verification of biological indicators by means of a normal sterilisation or decontamination machine comprising the technical features disclosed in one or more of the appended claims.

In a first aspect of the invention, the aforesaid objects, and others that will become more apparent further below, are achieved by an integration kit for a normal sterilisation or decontamination machine, according to what is described in claim 1.

Advantageous aspects are described in the dependent claims 2 to 10.

In a second aspect of the invention, the aforesaid objects are achieved by an integration method for a normal sterilisation or decontamination machine according to what is described in claim 11.

In a third aspect of the invention, the aforesaid objects, and others that will become more apparent further below, are achieved by a normal sterilisation or decontamination machine, according to what is described in claim 12.

Advantageous aspects are described in the dependent claim 13.

In a fourth aspect of the invention, the aforesaid objects are achieved by a method for characterising biological indicators used in processes of sterilisation or decontamination of different products according to what is described in claim 14.

Advantageous aspects are described in the dependent claim 15.

Additional features and advantages will become more apparent from the description of preferred, but not exclusive embodiments, of an accessory kit for a normal sterilisation or decontamination machine for production, illustrated by way of non-limiting example with the aid of the appended drawings, in which:

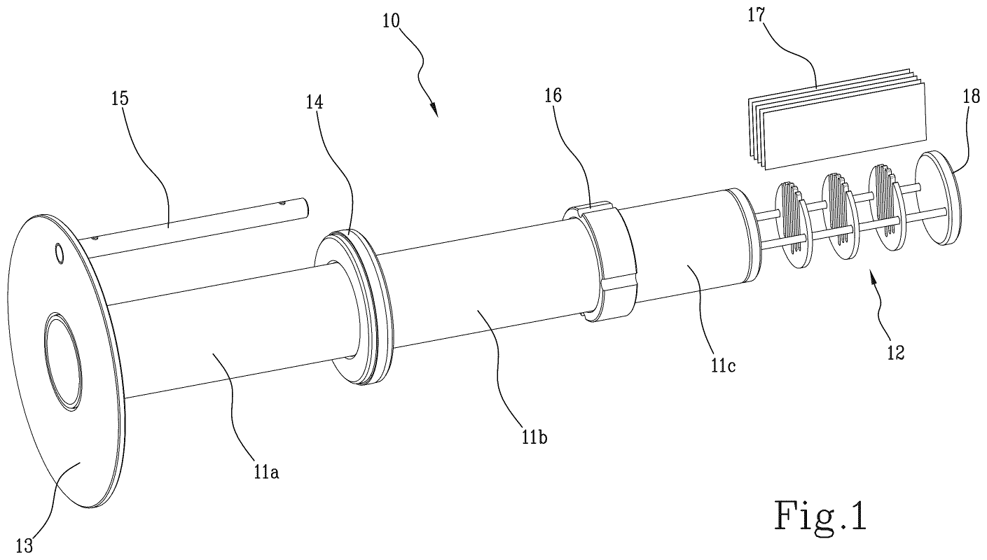

FIG. 1 shows an element on which the indicators according to the present invention are connected;

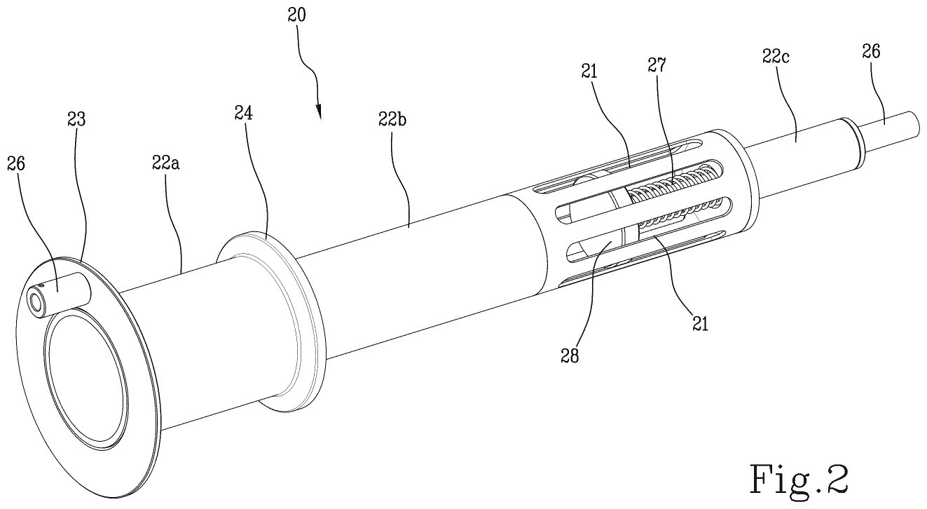

FIG. 2 shows a guide channel for the indicator loading element in FIG. 1;

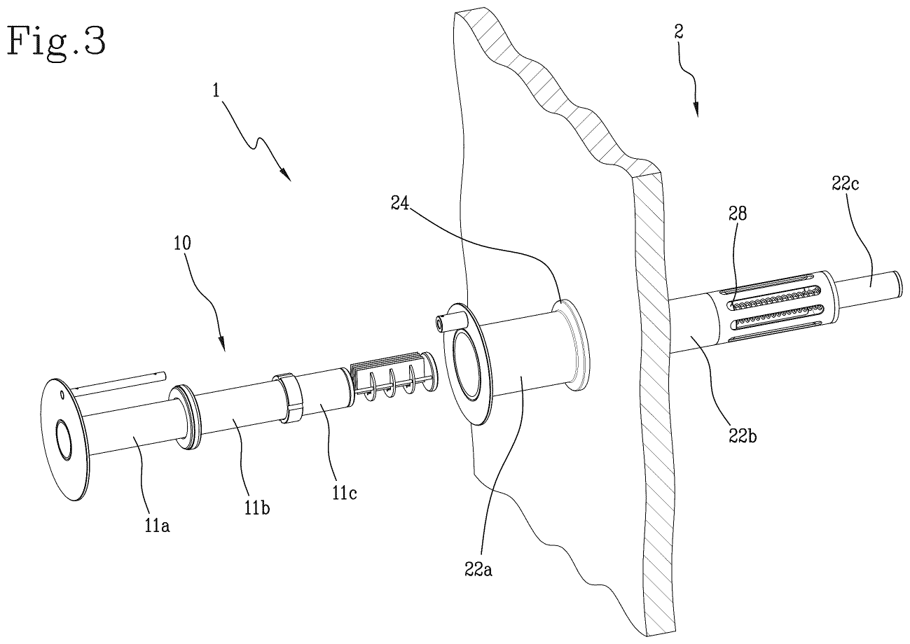

FIGS. 3 to 5 show the guide channel in use on a normal sterilisation or decontamination machine and the various steps of inserting the indicator loading element into the sterilisation chamber.

With reference to the appended figures, a sterilisation or decontamination machine comprising therein a sterilisation or decontamination chamber 2 is denoted in its entirety by the reference number 1.

The sterilisation or decontamination chamber is configured to receive therein the products to be sterilised during normal production through an access door (not shown in the figures).

During use, the chamber 2 is hermetically sealed off by the door and the sterilising agent (e.g., steam and/or sterilisation gas) is fed into the chamber 2 in a known manner.

The sterilisation chamber 2 can have a horizontal axis (for example it can be cylindrical or rectangular) or a vertical axis (like for example in small mechanical apparatus used in laboratories).

A first aspect of the invention relates to an integration kit or accessory device for the verification of biological indicators by means of a sterilisation or decontamination chamber.

In particular, the integration kit is installed on a normal sterilisation or decontamination machine 1, comprising a sterilisation or decontamination chamber 2 configured to contain therein the products to be sterilised and/or decontaminated.

The integration kit comprises an indicator loading element 10 which is insertable and removable from said sterilisation chamber 2 during a sterilisation process.

The indicator loading element 10 has a substantially longitudinal extent and can be inserted into the sterilisation chamber according to any orientation whatsoever with respect to the axis of the sterilisation chamber 2. For example, it can be inserted horizontally with respect to the floor on which the machine rests, in a direction orthogonal to the machine axis, or with any angle and/or position.

The biological indicator loading element 10 can be inserted through an access hole present in a wall of the normal sterilisation machine.

Preferably, the indicator loading element 10 can be inserted into the sterilisation chamber through an access hole present in the closure door of the normal sterilisation machine. In this manner, the element 10 will be located in the area of the protected environment of the machine. The indicator loading element 10, represented in FIG. 1, has a tubular shape with a longitudinal extent and comprises a first portion 11a, a central portion 11b and a second distal portion 11c.

During use, the first portion 11a will be outside the normal sterilisation or decontamination machine 1 whilst the central portion 11b and the second end 11c will be facing inside the sterilisation chamber 2 of the machine. As described further below, it will be possible to expose the biological indicators 17 selectively to the sterilising agent present inside the sterilisation or decontamination chamber 2 once the sterilisation conditions provided for have been reached.

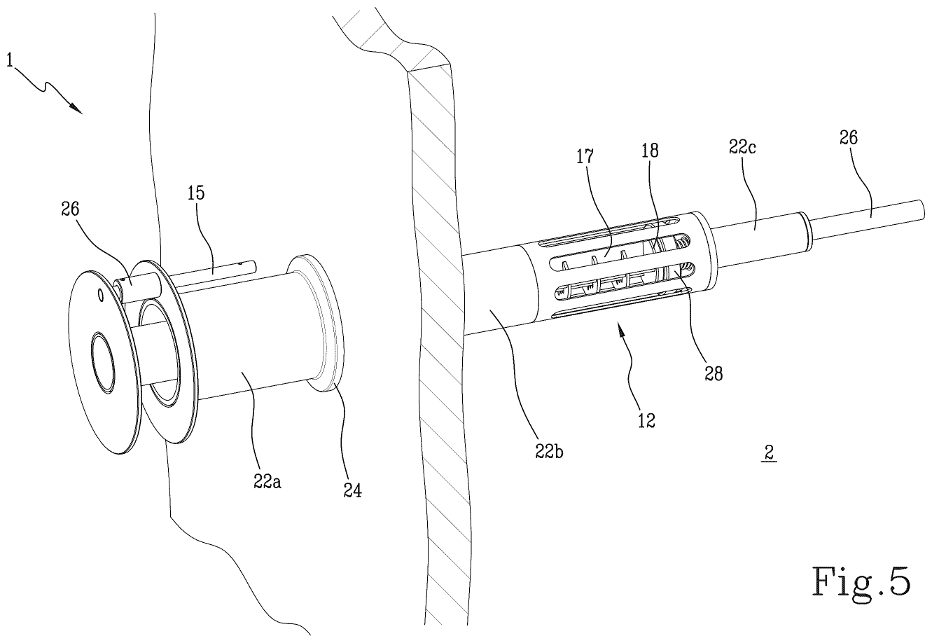

In proximity to the portion 11b, which is insertable into the sterilisation chamber, as shown in FIGS. 1 and 5, the indicator loading element 10 comprises one or more slots 12 or other housings configured to removably accommodate one or more biological indicators 17 to be selectively exposed to the sterilisation conditions present inside the sterilisation or decontamination chamber 2.

The indicator loading element 10 comprises an element or ring 13 configured to prevent it, during insertion into the guide channel 20, from ending up completely therein, i.e. it functions as a first blocking element. The element 10 comprises a sealing element 14, having the same inner diameter as the first portion 22a of the guide channel 20, so that it can slide inside the guide channel 20, while maintaining the environment inside the sterilisation machine separate from the outside one. The sealing element 14 comprises a sealing gasket.

The integration kit also comprises a guide channel 20 for the indicator loading element 10, shown in FIG. 2, which is configured to receive, coaxially and removably therein, the indicator loading element 10.

The guide channel 20 has a tubular shape, which is complementary to the shape of the indicator loading element 10, extends longitudinally and is configured to be inserted, at least partially, into a normal sterilisation or decontamination machine, so as to have, during use, a portion 22b, 22c selectively exposed to the inner environment 2 of the sterilisation or decontamination chamber 1 and to receive (from outside the sterilisation or decontamination machine) the indicator loading element 10 coaxially therein.

The guide channel 20 has an element or ring 23 configured to prevent the indicator loading element 10, during the insertion of the latter into the guide channel 20, from ending up completely therein, i.e. it functions as a blocking element together with the ring 13. Furthermore, an element or ring 24 is present which is configured to prevent, during use, the guide channel 20 from ending up completely inside the sterilisation or decontamination machine on which it is installed. In other words, the element 24 ensures that the portion 22a of the guide channel 20 remains outside the wall of the sterilisation or decontamination machine 1.

The guide channel 20 comprises a first portion 22a projecting towards the outside of the sterilisation chamber 2 (when inserted inside a sterilisation or decontamination chamber through a through-hole) and a second portion 22b which, during use, extends inside the sterilisation or decontamination chamber 2 and is in fluid communication therewith so as to selectively allow the passage therefrom and thereto of a sterilising agent present inside the sterilisation or decontamination chamber 2.

Preferably, the first portion 22a has a larger diameter than the through-hole (and than the second end 22b), so as to prevent the whole guide channel 20 from being involuntarily inserted completely inside the sterilisation chamber.

The guide channel 20 can be removably installed on a sterilisation or decontamination machine 1 by means of suitable fastening elements or quick clamps.

A distal portion of the second portion 22b of the guide channel 20 is, during use, selectively in fluid communication with the inside of the sterilisation or decontamination chamber 2 by virtue of the presence in said second end of one or more longitudinal openings 21 configured to put the portion of said indicator loading element 10 which houses the biological indicators 17 in fluid communication with the sterilisation or decontamination chamber 2.

The integration kit for a normal sterilisation machine 1 according to the present invention comprises an indicator loading element 10 which is insertable into and removable from said sterilisation or decontamination chamber 2 following a sterilisation process and a bioindicator guide channel 20 configured to removably receive said indicator loading element 10 therein, the guide channel 20 having a first portion 22a connectable outside the sterilisation or decontamination chamber 2 through a through-hole and a second portion 22b, 22c which, during use, extends inside the sterilisation or decontamination chamber 2 and which can be selectively put in fluid communication therewith so as to allow the passage therefrom and thereto of a sterilising agent present inside the sterilisation or decontamination chamber 2.

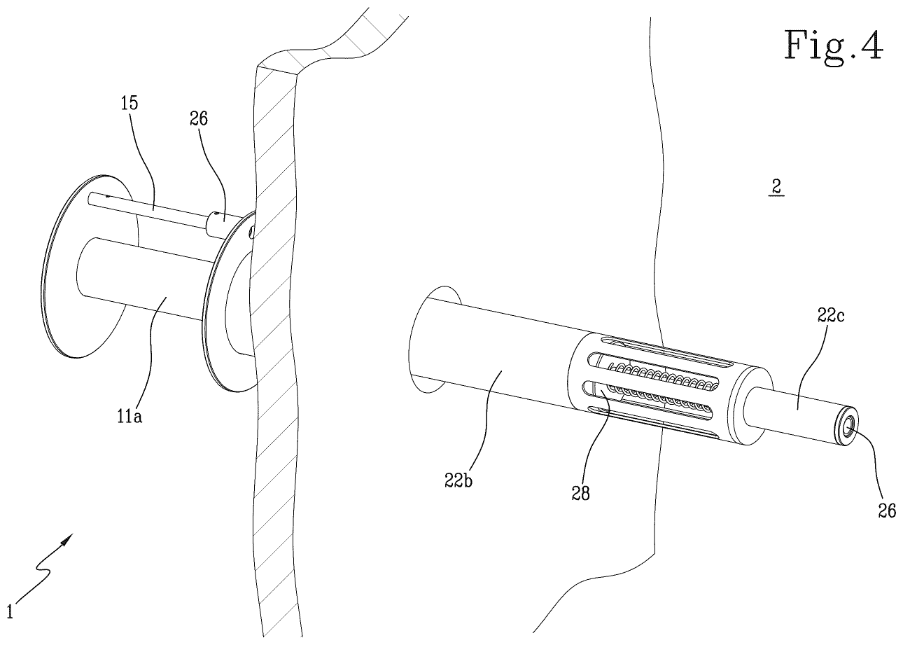

The integration kit comprises a first sealing means 14 and a second sealing means 28 which are mutually movable inside the guide channel 20 between a first position, in which the inner portion 22b (and the outer portion 22a) of the guide channel 20 is in contact with the environment outside the sterilising or decontaminating machine 1 and the inner portion 22b is isolated from the environment inside the machine 1 by the second sealing means 28, as illustrated in FIGS. 3 and 4; and a second position, in which the inner portion 22b of the guide channel 20 and the biological indicators 17 are in contact with the environment inside the sterilisation or decontamination chamber 2 (through the openings 21), and the inner portion 22b is isolated from the environment outside the normal sterilisation or decontamination machine 1 by the first sealing means 14. The environment inside the sterilisation chamber contains a sterilising agent.

Advantageously, the first sealing means 14 and the second sealing means 28 are mutually movable into a third position, in which the inner portion 22b and the outer portion 22a of the guide channel 20 are isolated both from the environment inside and from the environment outside the normal sterilisation or decontamination machine 1 by said first 14 and said second sealing means 28. In the third position, the inner portion 22b and the outer portion 22a of the guide channel 20 contain therein the sterilising or decontaminating agent present in the sterilisation or decontamination chamber 2 during the second position described above.

The mutual movement of the first sealing means 14 and second sealing means 28 allows the biological indicators 17 present on the indicator loading element 10 to be put selectively in fluid communication with the sterilising agent present inside the sterilisation or decontamination chamber 2 and makes it possible to prevent the inside environment from coming into contact with the outside environment during any phase of use of the sterilisation or decontamination machine 1.

The mutual movement of the sealing means 14 and 28 can be achieved either manually or by using a translation means. The outer portion 22a of the guide channel 20 comprises a means adapted to withdraw the sterilising agent present in the portion of the guide channel 20 comprised between the first sealing means 14 and the second sealing means 28 present in said third position, so as to avoid releasing it into the outside environment.

Before proceeding to extract the biological indicators 17, one proceeds to remove the sterilising or decontaminating agent present in an inner portion of said guide channel 20 comprised between the first sealing means 14 and the second sealing means 28. This removal can be achieved, for example, by blowing compressed air through access holes present in the portion 22a of the guide channel 20 (not present in the appended drawings). Advantageously, the environment inside and the environment outside the machine 1 never come directly into contact: first the outside environment is closed off (first position), then there is an opening towards the inside environment (second position).

Suitable retaining elements, not shown in the appended figures, allow the sealing means 14 and 28 to be placed in the positions described above.

Preferably, the first sealing means 14 is present on the indicator loading element 10 and the second sealing means 28 is present inside said guide channel 20.

Advantageously, the second sealing means 28 is present on one end of an elastic means 27 (for example a spring) present inside the guide channel 20 and is configured to compress towards a distal portion 22c during the insertion of the indicator loading element 10, so as to allow the sterilising agent to come selectively into contact with the biological indicators 17 through said openings 21.

During use, the guide channel 20 is inserted and fastened to a wall or the closure door of the sterilisation chamber 1, with the portion 22a protruding outside the sterilisation chamber 2 and the portions 22b and 22c present inside the latter.

It is possible to fit a normal sterilisation machine with a door comprising one or more through holes, each of which configured to receive therein a guide channel 20 according to the present invention.

During use, before the indicator loading element 10 is inserted, the inner part of the portions 22a and 22b of the guide channel 20 remains closed off and isolated from the environment inside the sterilisation chamber 2 by means of the shutter element 28, which, under the action of the elastic means 27, goes into the position shown in FIGS. 3 and 4.

FIGS. 3 to 5 illustrate the sequence of insertion of the indicator loading element 10 into the guide channel 20 in order to insert the biological indicators 17 into the sterilisation or decontamination chamber 2.

During insertion, the sealing element 14 initially internally reaches the position of the ring 24 and triggers the seal at the entry of the portion 22a of the guide channel 20 before the distal element 18 comes into contact with the shutter element 28 of the guide channel 20.

In this manner, the environment inside the sterilisation or decontamination chamber 2 is prevented from coming into contact with the environment outside the machine during the phase of exposure of the biological indicators 17.

When the indicator loading element 10 is inserted further towards the inside of the sterilisation or decontamination chamber 2, the distal element 18 pushes the shutter element 28, which will progressively compress the elastic means 27, thereby putting the indicators 17 in contact with the inside environment—and with the sterilising agent present therein—of the sterilisation or decontamination chamber 2, through the slots 21, as illustrated in FIG. 5.

During this insertion step, a guide rod 25 progressively and distally comes out of the guide channel 20, as shown in FIGS. 2, 4 and 5.

Once the indicators 17 have remained in contact with the environment of the sterilisation or decontamination chamber 2 for a given period of time, one proceeds to extract the indicator loading element 10 from inside the machine 1.

The extraction of the indicator loading element 10 takes place by bringing the sealing element 14 back into the position of the ring 23 of the guide channel 20. In this position, the sterilisation or decontamination chamber 2 of the machine remains closed off by the extension—by means of the action of the elastic means 27—of the shutter element 28 in the distal position of the portion 22b (FIGS. 3 and 4).

In this manner, the portions 22a and 22b of the guide channel 20 will be filled with the decontamination/sterilisation means present inside the sterilisation chamber, for example, hydrogen peroxide or other gases, and at the same time the inside of the sterilisation or decontamination chamber 2 will be isolated from the outside environment by virtue of the position of the shutter element 28 in the distal part of the portion 22b.

Before completely extracting the indicator loading element 10 from inside the guide channel 20, it may be necessary to carry out a step with compressed air on the environment present inside the portion 22a.

The step with compressed air can take place, for example, through an inlet hole and an outlet hole (not shown in the figures) through which to make a flow of compressed air enter and exit.

In this manner, the outside environment will never come into contact with the environment inside the sterilisation machine.

Advantageously, as shown in FIG. 2, the guide channel 20 comprises, in a first end thereof, a hollow tubular guide element 26 configured to allow the insertion therein, with the correct orientation, of said indicator loading element 10. The guide element 23 is configured to removably and coaxially house a protrusion 15 of the indicator loading element 10.

The guide channel 20 and the indicator loading element 10 have a circular cross section, or else a “D”-shaped cross section or one of any other shape, which is configured to prevent reciprocal rotation, so as to enable a correct orientation of the indicator loading element 10 and thereby assure the correct orientation and sliding of the indicator loading element 10 when it is inserted into the sterilisation chamber by means of the guide channel 20.

In a second aspect of the invention, the present invention relates to an integration method for a normal sterilisation machine 1 for production comprising a sterilisation chamber 2. The integration method comprises the steps of providing a sterilisation chamber 2 of a normal sterilisation machine for production configured to contain the products to be sterilised; obtaining a through-hole which passes through a wall or an access door of the sterilisation chamber 2, said through-hole being configured to removably receive a guide channel 20 for an indicator loading element 10 as described above.

It is possible to fit the through-hole with a sealed door or a valve that can be opened and closed from the outside.

The integration method comprises a step that comprises providing a bioindicator guide channel 20 configured to removably receive said indicator loading element 10 therein. The guide channel 20 has a first portion 22a connected outside the sterilisation chamber 2 through said through-hole and a second portion 22b, 22c which, during use, extends inside the sterilisation chamber 2 and is selectively in fluid communication therewith so as to selectively allow the passage therefrom and thereto of a sterilising agent present inside the sterilisation chamber 2.

In a third aspect of the invention, the present invention relates to a normal sterilisation machine 1 comprising—a sterilisation chamber 2 configured to contain the products to be sterilised;—a through-hole which passes through a wall or the access door of the sterilisation or decontamination chamber 2, configured to removably receive, from the outside of the machine 1, an indicator loading element 10 for loading indicators towards the inside of the sterilisation chamber 2,—a bioindicator guide channel 20 configured to removably receive an indicator loading element (10) therein, the guide channel 20 has a first portion 22a connected to the through-hole and a second portion 22b, 22c which extends inside the sterilisation chamber 2 and is in fluid communication therewith so as to allow the passage of a sterilising agent therefrom and thereto.

The guide channel 20 present during use in the normal sterilisation machine 1 comprises, in a distal portion, one or more openings 21 configured to selectively put said indicator loading element 10 in fluid communication with the sterilisation chamber 2.

It is possible to provide a sealed door or valve for said through-hole which can be opened and closed from the outside.

The bioindicator guide channel 20 and the indicator loading element 10 are as described above in relation to the first aspect of the present invention (integration kit).

In a fourth aspect of the invention, the present invention relates to a method for characterising biological indicators used in sterilisation processes, comprising the steps of:
providing a sterilisation chamber 2 of a sterilisation machine as described above, having a through-hole through which a guide channel 20 for biological indicators is removably inserted;-applying pre-set sterilisation conditions (e.g. with the use of a sterilising agent) to the sterilisation chamber 2;—introducing into the sterilisation chamber 2, through the guide element 20, an indicator loading element 10 so as to selectively allow the passage therefrom and thereto of a sterilising agent present inside the sterilisation chamber 2;
extracting, after a time set according to the treatment cycle to be carried out, said indicator loading element 10.

The indicator loading element 10 is inserted into a guide channel 20 as described above.

The method for characterising biological indicators comprises a step of removing the sterilising or decontaminating agent present at least in an inner portion of said guide channel 20 after exposing the biological indicators 17 to the sterilising agent present in the sterilisation chamber 2.

As the skilled person can observe, the present invention overcomes a large part of the drawbacks highlighted above with reference to the prior art.

The skilled person will be able, in order to satisfy specific needs, introduce modifications to the above-described embodiments of the invention and/or replace the elements described with equivalent elements, without going outside the scope of the appended claims.

1. An integration kit for a normal sterilisation machine (1) comprising a sterilisation chamber (2) configured to contain the products to be sterilised, the integration kit comprising:
an indicator loading element (10) which is insertable into and removable from said sterilisation chamber (2) following a sterilisation process; and
a bioindicator guide channel (20) configured to removably receive said indicator loading element (10) therein, the guide channel (20) having a first portion (22a) connectable outside the sterilisation chamber (2) through a through-hole and a second portion (22b, 22c) which, during use, extends inside the sterilisation chamber (2) and which can be selectively put in fluid communication therewith so as to allow the passage thereto and therefrom of a sterilising agent present inside the sterilisation chamber (2).

2. The integration kit according to claim 1, wherein said normal sterilisation machine (1) works at atmospheric pressure.

3. The integration kit according to claim 1, wherein said guide channel (20) comprises one or more openings (21) in said second portion, which are configured to put said indicator loading element (10) in fluid communication with the sterilisation chamber (2).

4. The integration kit according to claim 1, comprising a first sealing means (14) and a second sealing means (28) mutually movable inside the guide channel (20) between
a first position, in which the inner portion (22b) of the guide channel (20) is in contact with the environment outside the sterilising machine (1) and said inner portion (22b) is isolated from the environment inside the machine (1) by said second sealing means (28) and
a second position, in which the inner portion (22b) of the guide channel (20) and the biological indicators (17) are in contact with the environment inside the sterilisation chamber (2), and said inner portion (22b) is isolated from the environment outside the normal sterilisation machine (1) by said first sealing means (14).

5. The integration kit according to claim 4, wherein said first sealing means (14) and said second sealing means (28) are mutually movable into a third position, in which the inner portion (22b) and the outer portion (22a) of the guide channel (20) are isolated from both the environment inside and the environment outside the normal sterilisation machine (1) by said first (14) and said second sealing means (28).

6. The integration kit according to claim 5, wherein said outer portion (22a) of the guide channel (20) comprises a means adapted to withdraw said sterilising agent present in the portion of the guide channel (20) comprised between said first sealing means (14) and said second sealing means (28) in said third position.

7. The integration kit according to claim 4, wherein said first sealing means (14) is present on said indicator loading element (10) and said second sealing means (28) is present inside said guide channel (20).

8. The integration kit according to claim 4, wherein said second sealing means (28) is present on an elastic means (27) of said guide channel (20) configured to compress towards a distal portion (22c) during the insertion of said indicator loading element (10), so as to allow the sterilising agent to come into contact with said biological indicators (17) through said openings (21).

9. The integration kit according to claim 2, wherein the guide channel (20) comprises, in a first end of said portion (22a), a guide element (26) configured to allow the insertion therein, with the correct orientation, of said indicator loading element (10).

10. The integration kit according to claim 1, wherein said indicator loading element (10) comprises, in the end facing inside the sterilisation chamber, when in use, a series of slots (12) or other housings configured to contain the biological indicators (17) to be exposed to the sterilisation conditions inside the sterilisation chamber (2).

11. An integration method for a normal sterilisation machine (1) comprising a sterilisation chamber (2), the method comprising the steps of:
providing (2) a normal sterilisation machine (1) configured to contain the products to be sterilised;
obtaining a through-hole which passes through a wall or a door of the sterilisation chamber (2), said through-hole being configured to removably receive a bioindicator guide channel (20) according to one or more of claims 1 to 10;
providing a bioindicator guide channel (20) according to one or more of claims 1 to 10, configured to removably receive an indicator loading element (10) therein, the guide channel (20) having a first portion (22a) connected outside the sterilisation chamber (2) through said through-hole and a second portion (22b, 22c) which, during use, extends inside the sterilisation chamber (2) and is selectively in fluid communication therewith so as to allow the passage thereto and therefrom of a sterilising agent present inside the sterilisation chamber (2).

12. A normal sterilisation machine (1) comprising:
a sterilisation chamber (2) configured to contain the products to be sterilised;
a through-hole which passes through a wall or the door of the sterilisation chamber (2), said through-hole being configured to removably receive, from the outside of the machine (1), an indicator loading element (10) for loading indicators towards the inside of the sterilisation chamber (2);
a bioindicator guide channel (20) is configured to removably receive an indicator loading element (10) therein,
the guide channel (20) having a first portion (22a) connected to said through-hole and a second portion (22b, 22c) which extends inside the sterilisation chamber (2) and is in fluid communication therewith so as to allow the passage of a sterilising agent therefrom and thereto.

13. The normal sterilisation machine (1) according to claim 12, wherein said guide channel (20) comprises one or more openings (21) configured to put said indicator loading element (10) in fluid communication with the sterilisation chamber (2).

14. A method for characterising biological indicators used in product sterilisation processes, comprising the steps of:
providing a normal sterilisation machine (1) configured to contain the products to be sterilised, in accordance with claim 12, having a through-hole through which a guide channel (20) for biological bioindicators is removably inserted;
applying the pre-set sterilisation conditions to the sterilisation chamber (2);
introducing into the sterilisation chamber (2), through said guide channel (20), an indicator loading element (10) so as to selectively allow the passage therefrom and thereto of a sterilising agent present inside the sterilisation chamber (2);
extracting, after a time chosen according to the treatment cycle to be carried out, said indicator loading element (10).

15. The method for characterising biological indicators according to claim 14, comprising a step of removing the sterilising or decontaminating agent present at least in an inner portion of said guide channel (20) after exposing the biological indicators (17) to the sterilising agent present in the sterilisation chamber (2).
